(19) United States
(12) Patent Application Publication
(10) Pub. No.: US 2013/0071848 A1
(43) Pub. Date: Mar. 21, 2013

(54) One-step method for quantitative determination of uracil in DNA by real-time PCR

(71) Applicants: Beata G. Vertessy, Budapest (HU); Andras Horvath, Budapest (HU); Vince Grolmusz, Budapest (HU)

(72) Inventors: Beata G. Vertessy, Budapest (HU); Andras Horvath, Budapest (HU); Vince Grolmusz, Budapest (HU)

(21) Appl. No.: 13/237,987

(22) Filed: Sep. 21, 2011

(51) Int. Cl. C12Q 1/68

(52) U.S. Cl. CPC C12Q 1/686
USPC 435/6.12

(57) ABSTRACT

Uracil may occur in DNA due to either cytosine deamination or thymine replacing incorporation. Its quantitative characterization is important in assessing DNA damages in cells with perturbed thymidylate metabolism or within different DNA segments involved in immunoglobulin gene diversification. The archaeal DNA polymerase from Pyrococcus furiosus binds strongly to the deaminated base uracil and stalls on uracil-containing templates. Here, we present a straightforward method for quantitative assessment of uracil in DNA within specific genomic segments. We use wild type Pyrococcus furiosus polymerase in parallel with its point mutant version which lacks the uracil-binding specificity on synthetic and genomial DNA samples to quantify the uracil content in a single-step real-time PCR assay. Quantification of the PCR results is based on an approach analogous to template copy number determination in comparing different samples. Data obtained on synthetic uracil-containing templates are verified by direct isotopic measurements.

### INTRODUCTION

Genetic information is primarily stored within the varied sequences of the four well-known bases of DNA: adenine, guanine, cytosine, and thymine. In addition, the occurrence of other specifically modified bases becomes more and more appreciated as these are involved in diverse regulatory (i.e. epigenetic) and damage response pathways. Examples include cytosine methylation, related to the regulation of gene expression in eukarya and removal of infectious DNA in bacteria (1), as well as modification of the common bases via oxidation, alkylation or spontaneous deamination. Effects of reactive oxygene species (ROS) can result in 8-hydroxyguanine formation (2). S-adenosylmethionine (SAM) that provides the methyl group for cytosine methylation has a strong spontaneous transfer potential that can also convert adenine to 3-methyladenine (3). The most frequent spontaneous base modification is cytosine deamination that results in uracil appearance in DNA (4,5). Tautomers of guanine and adenine can produce xanthine and hypoxanthine, respectively (6). In some cases, enzyme catalyzed base modifications also present DNA damage signal and induce signal transduction. During B-cell maturation, somatic hypermutation and class switch recombination are known to be initiated by activation induced deaminase (AID) that catalyzes cytosine deamination in specific loci resulting in uracil bases. This is followed by a perturbed DNA repair action resulting in hypermutation and double strand breaks, respectively (7).

Besides cytosine deamination, uracil can accumulate in DNA if dTTP biosynthesis is disturbed. Abnormally elevated dUTP/dTTP ratios will lead to thymine replacing uracil incorporation since most DNA polymerases do not distinguish between thymine and uracil (4,8). Such perturbed dUTP/dTTP nucleotide pool ratios will be produced in cells where key enzymes of de novo thymidylate biosynthesis do not function properly. Among these enzymes, thymidylate synthase and dihydrofolate reductase catalyse methylation of the obligate precursor dUMP producing dTMP. The enzyme dUTPase converts dUTP into dUMP thereby provides input into dTMP synthesis and also eliminates dUTP from the dNTP pool (9). Absence or inhibition of these enzymes leads to drastic increase of uracil level in DNA (10-13).

Uracil appearance in DNA activates base excision repair (BER) (14), in which uracil recognition is carried out by uracil DNA glycosylase (UDG). Among the members of UDG family, UNG plays a major role in uracil recognition and removal (15). UDG removes the uracil base leaving an abasic (apurinic/apyrimidinic) (AP) site that is further cleaved by AP endonuclease. Repair is completed by DNA polymerase and ligase. Archaea possess an additional mechanism to avoid uracil accumulation in DNA: archaeal family B DNA polymerases possess a specific binding site that recognizes deaminated bases during replication (16,17). DNA synthesis will be stopped if uracil or hypoxanthine is detected in DNA. DNA polymerase stalling results in the accumulation of DNA repair enzymes around the deaminated base position (18,19). If dTTP biosynthesis is perturbed and dUTP concentration reaches high levels in the overall dNTP pool, deoxyuridine is repeatedly incorporated during both replicative and repair synthesis. Hyperactivated BER may initiate the so called thymine-less cell death by the frequent DNA cleavages (20). Since thymine-less cell death may be independent from p53 pathways (21,22); it has been considered as a promising anti-cancer therapeutic strategy. Several anti-cancer chemotherapeutic agents, such as 5-fluorouracil (5FU), 5-fluoro-2′-deoxyuridine (5FdUR), methotrexate and raltitrexed are widely used in the clinic to inhibit thymidylate synthase and dihydrofolate reductase, respectively (23-26).

Measurement of uracil content of DNA is an interesting challenge, because it is difficult to distinguish between uracil and thymine. Some approaches analyze nucleoside composition by liquid chromatography-tandem mass spectrometry (LC MS/MS) after DNA hydrolysis (27). In other methods, uracil DNA glycosylase is used as a sensor for uracil bases and uracil moieties are detected by gas chromatography-mass spectrometry (GC-MS) (10,13,28) or HPLC MS/MS (29) after derivatization. Furthermore, AP sites generated by UDG treatment can also be detected by the specific reactions with [¹⁴C]methoxyamine (30) or by aldehyde reactive probe (ARP) (11,31,32). Extent of DNA fragmentation at AP sites can be detected in single cell gel electrophoresis or DNA fractionation (12,33,34) after UDG treatment. An important limitation of single cell gel electrophoresis is the absence of comparable values for other assays regarding the exact amount of uracil, and quantitative analysis is also complicated. UDG-based MS and ARP assays require multiple steps and complex instrumentation for uracil detection. Recently, quantitative real-time PCR techniques to detect several DNA modifications have been reported (cf below); however, no such technique is yet available for quantifying uracil in DNA.

Real-time PCR-based assays for modified DNA bases such as oxidated bases, thymidine dimers, abasic sites, DNA adducts or methyl-cytosine rely on the altered interaction of the thermostable DNA polymerase from Thermophilus aquaticus (Taq pol) with modified bases. Taq polymerase can be directly inhibited by the modification (35-40), or the modified bases can be further processed to inhibitory complexes (41). In other types of detection, further modified bases having altered hybridization preferences cannot form primer template complexes (42).

In this study we report a new direct approach to quantify uracil in DNA by real-time PCR using the B-type DNA polymerase of Pyrococcus furiosus (Pfu pol). Binding of deaminated bases to a specific binding site within the Pfu pol enzyme blocks polymerization and extension of the primer-template junction is limited to only to four basepairs upstream the deaminated base (18,19,43). A point mutation within this binding site from valine to glutamine at position 93 disables uracil and hypoxanthine recognition (16,17). We used wild type (Pfu WT-pol, the uracil sensor) and point mutant (Pfu V93Q-pol, the reference) enzymes to directly detect uracil in DNA. The difference between the productivity of the two enzymes is evaluated to quantify the amount of uracil in the DNA sample. The assay is verified by independent isotopic quantification experiments. We also present the data obtained by this novel assay on several genomic DNA samples from cells with potentially altered uracil-DNA metabolism such as BL21(DE3) ung-151 and CJ236 (dut-1 ung-1) E. coli strains (11) and ung^{(-/-)} mouse embryonic fibroblast (MEF) cells (34) in the absence and presence of 5FdUR, as well as the respective wild type controls. Our real-time PCR-based assay possesses the benefits associated with similar assays for other modified DNA bases. It allows the analysis of site specific events or the heterogeneity of uracil distribution within the genome. The assay may also provide insights into the global effects of factors influencing uracil accumulation in DNA by a comparative analysis of the different effects.

Materials and Methods

Artificial Template Synthesis and Purification

For the synthesis of artificial templates pBS-dutP plasmid was used that was constructed as follows: PCR was performed with the dproFw and dproRev primers and Phusion High Fidelity DNA Polymerase (Finnzymes) on Drosophila melanogaster genomial DNA. The product was phosphorylated by Polynucleotide Kinase (Fermentas) and ligated into EcoRV (New England Biolabs) digested pBlueScript SK+ plasmid (Stratagene). The insert was found to be the same orientation as the plasmid.

Synthesis of dUMP containing DNA fragments. The PCR was performed by RedTaq DNA polymerase (Sigma-Aldrich) in 50 μl final reaction volume containing the buffer supplied by the manufacturer, the pBS-dutP plasmid DNA as template, 200 nM of each pBS-Fw and pBS-Rev primers, 200 μM of each dATP, dGTP, dCTP and 200 μM of dUTP and dTTP mix (Fermentas) to have the following dUTP/dNTP ratios: 0.1, 0.2, 0.4, 0.6, 0.8, 1, 2, 3, 4, 5% in the different samples. The reaction conditions were as follows: 95° C. at 30 s, followed by 35 cycles of 95° C. 30 s, 55° C. 60 s, 72° C. 60 s and finally an extension at 72° C. at 10 min.

Synthesis of radio-labeled dUMP containing DNA fragments. The reaction setup was the same as in the case of unlabeled DNA fragments except for the applied dUTP contained 1 or 10% tritium labeled dUTP [5-³H] (American Radiolabelled Chemicals Inc.). 10% dUTP[5-³H]/dUTP: samples with 0.1 and 0.2% dUTP/dNTP ratios; 1% dUTP[5-³H]/dUTP: samples with 0.4, 0.8, 1, 2, 3, 4 and 5% dUTP/dNTP ratios.

Purification. The PCR products were separated on 1% agarose gel, the fragments were cut and purified by Qiagen Gel extraction Kit following the instructions of the manufacturer. DNA concentration was determined from UV absorbance spectrum using a Nanodrop ND-1000 Spectrophotometer (Thermo Scientific).

Physiological DNA Isolation and Purification

Escherichia coli plasmid DNA. The pBS-dutP plasmid was transformed into XL1-blue, BL21(DE3)ung-151 and CJ236 (dut-1 ung-1) E. coli strains. The cell cultures were grown overnight in Luria broth (LB) media at 37° C., and the plasmids were purified by Qiagen Plasmid Miniprep Kit.

Escherichia coli genomial DNA. XL1-blue, BL21(DE3)ung-151 and CJ236 E. coli strains were propagated in LB media at 37° C. and were harvested at the early log phase (OD₆₀₀=0.5) or at saturated phase (overnight culture). BL21(DE3)ung-151 cells grown in the presence of 30.7 or 61.3 μM 5-fluoro-2′-deoxyuridine (5FdUR) (Sigma-Aldrich) were harvested at the early log phase. Genomial DNA was purified by MasterPure DNA Purification Kit (Epicentre) following the instructions of the manufacturer. The DNA was digested by NdeI restriction enzyme (New England Biolabs) and was separated on 1% agarose gel. To concentrate the fragment of interest which corresponds to the DNA fragments for which the detection primers were designed, a fragment at 5 kb was purified with Qiagen Gel Extraction Kit.

Mouse Embryonic Fibroblast (MEF) genomial DNA. Wild type and ung^{(-/-)} MEF cells (34) were a generous gift from Dr. Hilde Nilsen, University of Oslo. MEF cells were cultured in DMEM+F12 HAM Mix (Sigma) supplemented with 10% Fetal Bovine Serum (Gibco), 1% Penicillin+Streptomycin (Gibco), 0.5 mM Sodium Pyruvate (Gibco), 1% 100× Non-Essential Amino Acids (Gibco). To obtain confluent cells 10⁶ cells/T-75 flask were seeded. Wild type and ung^{(-/-)} cells were grown either in the absence of 5FdUR or in the presence of 100 μM and harvested after 4 days, at which time confluency was observed in samples without 5FdUR. Genomial DNA was purified by MasterPure DNA Purification Kit (Epicentre). DNA was digested by EcoRI restriction enzyme (New England Biolabs) and separated on 1% agarose gel. To concentrate the DNA fragmens for which the detection primers were designed, a fragment at 5 kb was purified with Qiagen Gel Extraction Kit.

Viability Assay of MEF Cells

Wild type and ung^{(-/-)} MEF cells were seeded onto 96-well plates in DMEM+F12 HAM Mix (Sigma) supplemented with 10% Fetal Bovine Serum (Gibco), 1% Penicillin+Streptomycin (Gibco), 0.5 mM Sodium Pyruvate (Gibco), 1% 100× Non-Essential Amino Acids (Gibco). 2000 cells were seeded in a well containing 100 μl medium. After 4 days 10 μl AlamarBlue (Biosource) was added, incubated for 3 hours at 37° C. and fluorescence was measured by Victor2 140 Multilabel counter at 485 nm excitation and 530 nm emission. Samples in triplicates were treated with different concentration of 5FdUR (0, 1, 10, 20, 50, 100, 250, 500, 1000 nM) and viability was assessed. Measurements were repeated three-times. Fluorescence intensity values measured from the wells was normalized to the intensity detected in samples grown in lack of 5FdUR.

Radioisotope Labeled Deoxyuridine Assay

The DNA fragments synthesized in the presence of dUTP[5-³H] were dissolved in 5 ml Optifluor Liquid Scintillation Counting Coctail (ParkinElmer) and tritium activity was measured in Wallac 1049 DSA Liquid Scintillation Counter. Calibration curve was determined from serial dilutions of the dUTP[5-³H] solution.

Quantitative Real-Time PCR

Synthetic and physiological samples purified from agarose gels were applied in series of either 2-fold or 10-fold dilution steps. In the case of genomial derived DNA, the samples were diluted 2-fold because of the narrow linear range of the Cq-log(template concentration) plots. Depending on the linearity range, serial dilutions consisted of 5-10 steps. Reaction mixture was in a final 10 μl volume and contained 0.05 units of PfuTurbo® Hotstart (Pfu WT-pol) or PfuTurbo® Cₓ Hotstart (Pfu V93Q-pol) DNA polymerase (Stratagene), 0.175 μM of each primers (Eurofins MWG Operon, HPLC grade), 200 μM of each dNTP (Fermentas), 0.5 μl EvaGreen 20× (Biotium), 30 nM Passive Reference Dye (Stratagene) and 1 μl of DNA template from one of the dilution series. Reactions were performed in the reaction buffer provided by the manufacturer for the PfuTurbo® Hotstart or PfuTurbo® Cₓ Hotstart DNA polymerases (Stratagene). Nuclease-free water (Ambion) was used for the reactions and for sample dilutions. Real-time PCR reactions were performed in Stratagene MX3000P™ (Agilent Stratagene) instrument in 96-well plate. pUbsd-Fw and pUbsd-R544 or pUbsd-R1057 primers were used for measurements on artificial templates and plasmid DNA amplifying 544 or 1057 bp long sequence. In the case physiological samples, primers gdh656_Fw and gdh656_Rev were used for measuring the uracil content of E. coli genomial DNA, whereas primers MEFdut_{—}1168Fw and MEFdut_{—}1168Rev were used for measuring the uracil content of mouse fibroblast genomial DNA. Reaction conditions were: 95° C. for 2 min, 40 cycles of 95° C. for 15 s, 57° C. for 10 s, 72° C. for 50 s in the case of 544 bp long sequence amplification and 70 s in the case of longer amplicons. Melting point of the products was measured after a final denaturation at 95° C. for 1 min by heating from 55° C. to 95° C. Absence of ascpecific products was also confirmed on 1% agarose gel. Non-template control was measured in all cases. Evaluation of the reaction was carried out on MxPro v4.01 software, using reference dye normalization. Cq values were determined after the manual setting of threshold level in the early exponential phase of the PCR amplification. Cq values were plotted with the logarithmic scale of dilution steps and linear was fitted (R²>0.9). Efficiency was calculated from the following equation: E=10^{(-1/M)}−1, where M is the slope of the linear curve and E is the efficiency. The Cq values for Pfu WT-pol (Cq_{WT}) were plotted against the corresponding Cq values for Pfu V93Q-pol (Cq_{V93Q}) in case of both the reference and uracil substituted samples; data were fitted by linear equations. At each measured Cq_{Wt} value the shift between the linear curves of the reference and the putatively uracil containing samples was determined. The values of calculated uracil content are represented as mean±standard error of mean (SEM), derived from 3-10 independent measurements.

Results

Principles of the Pfu DNA Polymerase Based Deoxyuridine Detection in the DNA

In an arbitrary DNA segment, let U=(number of uracil moieties)/(total number of all bases). Assuming that the uracil moieties are randomly distributed within the DNA segment, this U value also equals the probability that the base of any particular mononucleotide is uracil. Also, (1−U) is equal to the probability that any particular mononucleotide unit is not dUMP. Let S be the number of mononucleotide units in the segment to be amplified by PCR. Necessarily, S will be strictly defined by the primers and the DNA sequence. In practice, this requires appropriate selection of primers and lack of low complexity regions in the DNA segment to be amplified. Our considerations fully apply only if the selectivity of the PCR reaction is absolute, ie. only the template segment with S base length defined by the primers is being amplified during the PCR reaction. Random distribution of the uracil moieties implies that presence or absence of uracil in any mononucleotide unit can be considered as independent events. Therefore, the probability (P_{S,U}) that a DNA segment of S base length in the DNA sample characterized by U uracil content does not contain any uracil moiety can be described as the arithmetic product of the individual probabilities for each mononucleotide units not being uracil along the sequence S:

P_{S,U}=(1−U)^{S} (1)

In a typical DNA sample used for real-time quantitative PCR, there will be a definite number of templates, possessing the targeted S-base length sequence defined by the primers of the PCR reaction. Let N be the number of the S-base length template in the given DNA sample. If U characterizes the uracil content of this template DNA segment as defined above and if the distribution of uracil moieties is random within the template, then according to probability theory, the expected number of S-length templates that do not contain any uracil moieties, N_{U-free} will be:

N_{U-free}=N·(1−U)^{S} (2)

A DNA polymerase that does not distinguish between uracil and thymine will therefore amplify all templates (N); however, another DNA polymerase that cannot use uracil-substituted templates will amplify only uracil-free templates (N·(1−U)^{S}). Provided that such uracil-insensitive and uracil-sensitive DNA polymerases are available that work basically according to the same mechanism with the single alteration regarding the uracil discrimination (cf. Pfu WT-pol and Pfu V93Q-pol as described above), the uracil content of the PCR reaction template can be characterised by comparing the productivity of the two enzymes. Pfu WT-pol is known to bind uracil moieties in single-stranded DNA with a high affinity (FIG. 1A) (17,19). Hence, this polymerase stalls on uracil containing templates, and replication may occur only on segments that does not contain any uracils. The probability P_{S,U}=(1−U)^{S}, as defined above, therefore also defines the probability that Pfu WT-pol can replicate a DNA segment of S base length in the DNA sample characterized by U uracil content. This probability also describes the proportion of templates without uracils (N·(1−U)^{S}) in that DNA sample. In a uracil-free reference sample, the concentration of template without uracils equals the total template concentration ((N·(1−U)^{S})=N), since U=0 in this reference sample. The V93Q mutant species of Pfu pol, however, can replicate all templates (N) regardless of the uracil content (FIG. 1B) (16,17). FIG. 1C shows that using both enzymes, very similar efficiency values (E=10^{(-1/slope)}−1) (44) can be determined for templates either lacking or containing uracil moieties, indicating that i) amplification of uracil-free templates by Pfu WT-pol is not affected by the uracil content of other DNA fragments in the examined concentration range of the template; ii) there is no partial inhibitory effect on the activity of the Pfu V93Q-pol by the uracil bases in the DNA. In our method, we exploit the difference between the selectivity of the wild type and V93Q mutant Pfu polymerase enzyme species. Therefore, the ratio of uracil-free as compared to total template concentration can be defined by the portion of templates amplifiable by Pfu WT-pol compared to the total template number:

$${ratio}_{( U - \frac{free}{total} )} = \frac{N \cdot {( 1 - U )}^{S}}{N} = {( 1 - U )}^{S} \qquad ( 3 )$$

In the experimental setup, both wild type and V93Q enzymes are used on a uracil-containing and a uracil-free reference samples. In any given sample, Pfu V93Q-pol allows determination of the quantification cycle number at the threshold fluorescence (Cq value) that is representative for every template of that sample regardless of uracil content. Template concentration determined by the Pfu V93Q-pol serves as a control to which Pfu WT-pol measurements can be normalized in uracil containing and reference samples. Such a system is fully analogous to the classic comparative real-time PCR measurements of template copy numbers (cf. Supplementary material). In our system, therefore, the usual ΔΔCq method can be used for evaluation of the experimental data and to determine the ratio of uracil-free and all templates (45):

$${ratio}_{( \frac{total}{U} - free )} = \frac{{( 〚 {E}_{WT} + 1 ) 〛}^{{C}_{{q}_{WTura}} - {Cq}_{WTref}}}{{( {E}_{V93Q} + 1 )}^{{C}_{{q}_{V93Qura} - {C}_{{q}_{V92Qref}}}}} \qquad ( 4 )$$

where ratio_{(total/U-free)} is equivalent to the reciprocal value of ratio_{(U-free/total)}, as defined in equation (3), E_{WT} and E_{V93Q} stand for the efficiency of amplification performed by Pfu WT- or V93Q-pol, respectively, Cq_{WTura}, Cq_{V93Qura}, Cq_{WTref} and Cq_{V93Qref} represent the Cq values determined from the uracil-containing or the reference samples by Pfu WT- or V93Q-pol, respectively.

In a more direct way, to describe the effect of the uracil containing DNA samples compared to a reference, we can plot Cq values determined by Pfu WT-pol as a function of the Cq values determined by Pfu V93Q-pol as shown in FIG. 1D. In this representation, the Cq_{WT} vs. Cq_{V93} curve fitted on data obtained from dilution series of the uracil-containing samples is characteristically shifted along the Cq_{WT} axis as compared to that of uracil-free sample. This shift (ΔR) allows the direct demonstration of the inability of the wild type enzyme to replicate the uracil containing templates from the measured Cq values. ΔR can be expressed as the difference between the intercepts of the uracil containing and the reference samples in the Cq_{WT} axis. Since ΔR is a Cq difference along the Cq_{WT} axis, the ratio of the uracil-free (N·(1−U)^{S}) and all (N) templates can also be calculated as follows:

$${ratio}_{( \frac{total}{U} - free )} = {( {E}_{WT} + 1 )}^{Δ R} \qquad ( 5 )$$

In case of our measurements, the values of ratio given from (4) and (5) are identical; furthermore ΔR can be derived from ΔΔCq (cf. Supplementary material), therefore the more simple equation (5) can be used for evaluation of experimental data. Uracil content in the template can be calculated from the determined (N·(1−U)^{S}) and (N) ratio (reciprocal ratio value of equations (4) and (5)) according to equation (1):

$$U = 1 - {ratio}_{( U - \frac{free}{total} )}^{\frac{1}{S}} \qquad ( 6 )$$

U value is representative only for the DNA fragment designed by the primers used in the real-time PCR reaction.

Effects of Uracil Content of DNA and Amplicon Size on the U-Sensitive qPCR Assay

We synthesized artificial templates for calibration of our assay using Taq polymerase in the presence of different amount of dUTP in the reaction mixture. Since Taq polymerase is not selective for uracil residues in DNA (45,46), we assumed that the applied dUTP concentration will be proportional to the ratio of dUMP residues appearing in the synthesized DNA. Dilution series of the artificial templates were applied in real-time PCR using the uracil-sensitive Pfu WT- and the insensitive V93Q-pol. Reaction conditions were set to amplify 544 and 1057 bp products defined by the primers. Corresponding Cq values were determined and plotted as described above for both 544 and 1057 bp DNA fragments (FIG. 2A). In case of longer template, a higher shift can be observed between the linear curves of uracil-free reference and uracil-containing sample. This observation was in agreement with our expectations, since the longer the template, the higher the probability that it contains at least one uracil moiety. Therefore, the amount of uracil-free templates that can be replicated by Pfu WT-pol will be decreased. Since the Cq shift of the Pfu WT-pol (AR) serves as the signal for the detection, the increased shift generated by a longer amplicon allows more sensitive and accurate detection of uracil. Therefore sensitivity can be increased by choosing the appropriate primers for amplifying longer DNA fragments. Plotting the measured Cq values of Pfu WT- and V93Q-pol amplification of the serial dilutions of different artificial templates reveals that the shift between the linear curves (ΔR) is independent of the template concentration and is proportional to the dUTP ratio present in the dNTP pool used for the synthesis of the artificial template (FIG. 2B).

Validation of the U-Sensitive qPCR Assay

For a direct determination of the amount of uracil in the artificial templates synthesized in the presence of dUTP by Taq polymerase, we included ³H-labelled dUTP in the dNTP pool. From the detected radioactivity of the purified DNA, the amount of incorporated dUMP was calculated and compared to the values calculated from the real-time PCR data using Pfu DNA polymerases. As reference for evaluation of the real-time PCR data, we used the template synthesized by Taq in the presence of 0% dUTP/dNTP ratio assuming that (N·(1−U)^{S})=N in this sample. The amount of uracil calculated from the ΔR of the real-time PCR method showed good agreement with the data obtained from the isotope measurement (FIG. 2C, Table 1). Amounts of uracil determined from the same template amplifying 544 bp and 1057 bp products in real time-PCR also showed agreement in the overlapping range. The longer applicon provided data with much less error in the lower range of uracil content.

These measurements indicated that optimization of template length, with regard to the anticipated uracil content in a sample to be measured, is essential for reliable measurements in the presently described assay. Longer amplicon size allows more sensitive uracil detection; however, high uracil content may also inhibit the PCR reaction completely, either due to the very low probability of uracil-free template that is available for the Pfu WT-pol, or due to suboptimal concentration of Pfu WT-pol compared to the abundant level of uracil residues in the reaction mixture. If the uracil content of a DNA template is high, shorter amplicon size is recommended. In the case of low uracil content, only significantly longer amplicons can produce reliable data.

Uracil Content of Plasmid DNA in E. Coli Cells

Using DNA template obtained from organisms with potentially perturbed dUTP/dTTP ratio or with defective uracil excision repair, we applied the method described in this study on physiological samples and we expected to observe uracil accumulation on the template segment defined by the real-time PCR primers. Our results are considered to be valid only within the DNA segment used as template for the real-time PCR. These observations can be generalized for the whole genome or the entire plasmid only with the assumption that uracil is evenly and randomly distributed in the DNA.

Plasmids were isolated from XL1-blue (wild-type), BL21(DE3) ung-151 and CJ236 (dut-1 ung-1) E. coli overnight cultures. Serial dilutions of plasmids were applied as a template for Pfu WT- and V93Q-pol in real-time PCR amplifying 1057 bp fragments. Plotting the Cq values determined in PCR reactions with Pfu WT- and V93Q-pol showed that there is no significant difference between samples purified from wild-type and ung-151 cells, but a large shift along the Cq_{WT} axis was observed in the case of the dut-1 ung-1 sample (FIG. 3A). For calculation of uracil content, plasmid purified from wild type E. coli was used as uracil-free reference. Uracil content of plasmid DNA calculated from the ΔR shift was below detection level in the ung-151 samples. More sensitive measurements using longer amplicon length may allow a higher resolution for the ung-151 sample. For the dut-1 ung-1 sample, the value determined in our assay was 5490±85 deoxyuridine/million dNMP, in agreement with earlier data from the literature (11).

Uracil Content of E. Coli Genomial DNA

Wild type, ung-151 and dut-1 ung-1 cells were grown in early log phase (OD₆₀₀=0.5) or saturated (overnight) phase and genomial DNA was purified. The large segments of the sample DNA not utilized as template in the PCR may inhibit the Pfu WT-pol catalyzed reaction, if the DNA sample contains high amount of uracil. In this case frequently appearing uracil residues in the non-template DNA can deplete Pfu WT-pol, and the polymerase concentration will be suboptimal. This problem can be diminished by separating and concentrating the fragments of interest. Therefore template fragments were enriched after restriction enzyme digestion and separation on agarose gel by isolating DNA at the appropriate band size. DNA purified from saturated phase wild-type E. coli was used as the uracil-free reference for determining the uracil content of different samples. In saturated phase cultures, results on ung-151 samples and dut-1 ung-1 samples were 90±61 and 8063±167 deoxyuridine/million dNMP, respectively (FIG. 3B). We conclude that similarly to data obtained on plasmid DNA samples, uracil content of DNA was under the detection limit in the case of ung-151 E. coli cultures in the saturated growth phase cultures, and the data on the double mutant dut-1 ung-1 samples are again in agreement with published results (11).

In early phase cultures, similar measurements indicated that the uracil content of DNA from the ung-151 sample becomes detectable as 537±37 deoxyuridine/million dNMP in DNA. When ung-151 cells were incubated in the presence of 30.7 and 61.3 μM 5FdUR, these values increased to 653±55 and 770±54 deoxyuridine/million dNMP, respectively. Genomial DNA from dut-1 ung-1 cells at early phase contained 6580±174 deoxyuridine/million dNMP (FIG. 3B). In conclusion, accelerated cell division in the early (logarithmic) cell growth phase allowed detectable uracil accumulation in the ung-151 deficient cells.

Uracil Content within Genomic DNA of Mammalian Cells with Perturbed dTTP Synthesis

We examined the accumulation of uracil in DNA in wild-type or ung^{(-/-)} mouse embryonic fibroblast (MEF) cells after 5FdUR treatment. DNA purified from untreated wild-type MEF cells was used as uracil-free reference. According to our observations, the uracil content of untreated wild-type and ung^{(-/-)} MEF cells were both below detection limit (FIG. 3C). Wild-type MEF cells treated with 100 μM 5FdUR showed the practically undetectable values of 40±38 deoxyuridine/million dNMP, whereas in the case of ung^{(-/-)} MEF cell line, data obtained were significantly higher: 341±87 deoxyuridine/million dNMP (FIG. 3C). These results show that UNG deficiency allows cells to accumulate uracil-containing DNA, in agreement with earlier published data (12). We also tested the viability of the wild-type and ung^{(-/-)} MEF cells as a function of 5FdUR treatment (FIG. 3D). In both wild type and ung^{(-/-)} MEF cells, 5FdUR treatment resulted in significant decrease in viability, with similar dose-response characteristics, although at the lowest drug concentrations, wild type cells seemed to be somewhat more sensitive.

Discussion

Quantitative determination of modified bases in DNA is of increasing importance for both assessment of DNA damages and analysis of epigenetic signaling. The method we present can be used to determine the number of uracil moieties within a defined DNA segment with a simple, quantitative and fast one-step method. Employment of the two Pfu DNA polymerases (wild type and V93Q mutant) on the same samples allows quantitative and comparable results. To determine absolute uracil content, there is no need for calibration with controls containing different amounts of uracil residues. Performance and reliability of the one-step qPCR-based method is shown on both artificial and physiological samples. Data obtained on artificial samples (synthesized using pre-defined composition of nucleotide triphosphate pools) by isotope-based highly sensitive quantification show good agreement with the data determined using the presently described novel method (cf. FIG. 2C and Table 1). In addition, data obtained on physiological samples using the presently described method are comparable with published data. Since other approaches in the literature were intended to determine absolute uracil content from the whole genome, reliable comparison requires the assumption that uracil content measured by our method in the specific regions are representative for the overall DNA. Data published for dut-1 ung-1 E. coli genomial DNA range from 3000-7700 (11) to 12200 uracil per million bases (10); similar to our results (6500-8000 uracil per million bases). We observed the tendency of uracil accumulation in DNA of UNG deficient E. coli cells if they were harvested at early exponential or saturated growth phase (cf. FIG. 3B, in agreement with (11)) indicating that exponential proliferation allows increased uracil accumulation in DNA. In the case of mammalian cells, our measurements also showed similar tendencies after 5FdUR treatment: we found increased amount of uracil in the genome of ung^{(-/-)} but not in wild-type MEF cells (FIG. 3C, in agreement with (12)). Similar results were also reported in human embryonic kidney (HEK) 293 cells expressing the specific UNG family inhibitor UGI where content of uracil was increased only in the DNA of UGI expressing cells but not in the control (13).

Induction of DNA damage in anti-cancer chemotherapies is a widespread strategy. One-third of anti-cancer drugs presently used in the clinics targets the thymidylate biosynthesis pathway and induces gross imbalances in nucleotide pools (fluoropyrimidines, methotrexate and derivatives) (23-26). One major pathway of the mechanism of action of these drugs is to drive the level of uracil moieties in DNA to an excessively high rate and thereby transform the base-excision repair into a hyperactive futile cycle. To evaluate efficacies of these drugs, the quantitative determination of uracil levels in DNA as induced by the drugs is of interest. Recent and previous studies (12,13) showed that UNG deficiency allows uracil accumulation in MEF cells; however, other factors showing variable expression in tumor cells involved in thymidylate biosynthesis and BER (47) can affect the efficiency of a chemotherapic treatment.

The potential multiple signaling roles of uracil in DNA, as addressed in recent publications, was a major driving force in our study to provide a feasible and quick method for assessing uracil-content in DNA. Uracil has been implicated in somatic hypermutation and class-switch recombination at special sites (7), moreover transcription coupled dUMP incorporation (48) can be responsible for the heterogeneity of uracil content in DNA. Our method is potentially applicable to assessing the uracil content in a segment-specific manner to trace genomic regions with elevated uracil content.

### REFERENCES

1. Weber, M. and Schubeler, D. (2007) Genomic patterns of DNA methylation: targets and function of an epigenetic mark. Curr Opin Cell Biol, 19, 273-280.
2. Fortini, P., Pascucci, B., Parlanti, E., D'Errico, M., Simonelli, V. and Dogliotti, E. (2003) 8-Oxoguanine DNA damage: at the crossroad of alternative repair pathways. Mutat Res, 531, 127-139.
3. Sedgwick, B., Bates, P. A., Paik, J., Jacobs, S. C. and Lindahl, T. (2007) Repair of alkylated DNA: recent advances. DNA Repair (Amst), 6, 429-442.
4. Pearl, L. H. and Savva, R. (1996) The problem with pyrimidines. Nat Struct Biol, 3, 485-487.
5. Lindahl, T. (1993) Instability and decay of the primary structure of DNA. Nature, 362, 709-715.
6. Kow, Y. W. (2002) Repair of deaminated bases in DNA. Free Radic Biol Med, 33, 886-893.
7. Stavnezer, J., Guikema, J. E. and Schrader, C. E. (2008) Mechanism and regulation of class switch recombination. Annu Rev Immunol, 26, 261-292.
8. Mosbaugh, D. W. (1988) Purification and characterization of porcine liver DNA polymerase gamma: utilization of dUTP and dTTP during in vitro DNA synthesis. Nucleic Acids Res, 16, 5645-5659.
9. Vertessy, B. G. and Toth, J. (2009) Keeping uracil out of DNA: physiological role, structure and catalytic mechanism of dUTPases. Acc Chem Res, 42, 97-106.
10. Blount, B. C. and Ames, B. N. (1994) Analysis of uracil in DNA by gas chromatography-mass spectrometry. Anal Biochem, 219, 195-200.
11. Lari, S. U., Chen, C. Y., Vertessy, B. G., Morre, J. and Bennett, S. E. (2006) Quantitative determination of uracil residues in Escherichia coli DNA: Contribution of ung, dug, and dut genes to uracil avoidance. DNA Repair (Amst), 5, 1407-1420.
12. Andersen, S., Heine, T., Sneve, R., Konig, I., Krokan, H. E., Epe, B. and Nilsen, H. (2005) Incorporation of dUMP into DNA is a major source of spontaneous DNA damage, while excision of uracil is not required for cytotoxicity of fluoropyrimidines in mouse embryonic fibroblasts. Carcinogenesis, 26, 547-555.
13. Luo, Y., Walla, M. and Wyatt, M. D. (2008) Uracil incorporation into genomic DNA does not predict toxicity caused by chemotherapeutic inhibition of thymidylate synthase. DNA Repair (Amst), 7, 162-169.
14. Baute, J. and Depicker, A. (2008) Base excision repair and its role in maintaining genome stability. Crit Rev Biochem Mol Biol, 43, 239-276.
15. Kavli, B., Slupphaug, G., Mol, C. D., Arvai, A. S., Peterson, S. B., Tainer, J. A. and Krokan, H. E. (1996) Excision of cytosine and thymine from DNA by mutants of human uracil-DNA glycosylase. Embo J, 15, 3442-3447.
16. Fogg, M. J., Pearl, L. H. and Connolly, B. A. (2002) Structural basis for uracil recognition by archaeal family B DNA polymerases. Nat Struct Biol, 9, 922-927.
17. Gill, S., O'Neill, R., Lewis, R. J. and Connolly, B. A. (2007) Interaction of the family-B DNA polymerase from the archaeon Pyrococcus furiosus with deaminated bases. J Mol Biol, 372, 855-863.
18. Greagg, M. A., Fogg, M. J., Panayotou, G., Evans, S. J., Connolly, B. A. and Pearl, L. H. (1999) A read-ahead function in archaeal DNA polymerases detects promutagenic template-strand uracil. Proc Natl Acad Sci USA, 96, 9045-9050.
19. Shuttleworth, G., Fogg, M. J., Kurpiewski, M. R., Jen-Jacobson, L. and Connolly, B. A. (2004) Recognition of the pro-mutagenic base uracil by family B DNA polymerases from archaea. J Mol Biol, 337, 621-634.
20. Goulian, M., Bleile, B. M., Dickey, L. M., Grafstrom, R. H., Ingraham, H. A., Neynaber, S. A., Peterson, M. S. and Tseng, B. Y. (1986) Mechanism of thymineless death. Adv Exp Med Biol, 195 Pt B, 89-95.
21. Munoz-Pinedo, C., Oliver, F. J. and Lopez-Rivas, A. (2001) Apoptosis of haematopoietic cells upon thymidylate synthase inhibition is independent of p53 accumulation and CD95-CD95 ligand interaction. Biochem J, 353, 101-108.
22. Munoz-Pinedo, C., Robledo, G. and Lopez-Rivas, A. (2004) Thymidylate synthase inhibition triggers glucose-dependent apoptosis in p53-negative leukemic cells. FEBS Lett, 570, 205-210.
23. Rich, T. A., Shepard, R. C. and Mosley, S. T. (2004) Four decades of continuing innovation with fluorouracil: current and future approaches to fluorouracil chemoradiation therapy. J Clin Oncol, 22, 2214-2232.
24. Van Triest, B., Pinedo, H. M., Giaccone, G. and Peters, G. J. (2000) Downstream molecular determinants of response to 5-fluorouracil and antifolate thymidylate synthase inhibitors. Ann Oncol, 11, 385-391.
25. Schilsky, R. L. (1996) Methotrexate: an effective agent for treating cancer and building careers. The polyglutamate era. Stem Cells, 14, 29-32.
26. Li, L., Connor, E. E., Berger, S. H. and Wyatt, M. D. (2005) Determination of apoptosis, uracil incorporation, DNA strand breaks, and sister chromatid exchanges under conditions of thymidylate deprivation in a model of BER deficiency. Biochem Pharmacol, 70, 1458-1468.
27. Kiljunen, S., Hakala, K., Pinta, E., Huttunen, S., Pluta, P., Gador, A., Lonnberg, H. and Skurnik, M. (2005) Yersiniophage phiR1-37 is a tailed bacteriophage having a 270 kb DNA genome with thymidine replaced by deoxyuridine. Microbiology, 151, 4093-4102.
28. Blount, B. C., Mack, M. M., Wehr, C. M., MacGregor, J. T., Hiatt, R. A., Wang, G., Wickramasinghe, S. N., Everson, R. B. and Ames, B. N. (1997) Folate deficiency causes uracil misincorporation into human DNA and chromosome breakage: implications for cancer and neuronal damage. Proc Natl Acad Sci USA, 94, 3290-3295.
29. Ren, J., Ulvik, A., Refsum, H. and Ueland, P. M. (2002) Uracil in human DNA from subjects with normal and impaired folate status as determined by high-performance liquid chromatography-tandem mass spectrometry. Anal Chem, 74, 295-299.
30. Liuzzi, M. and Talpaert-Borle, M. (1985) A new approach to the study of the base-excision repair pathway using methoxyamine. J Biol Chem, 260, 5252-5258.
31. Kubo, K., Ide, H., Wallace, S. S. and Kow, Y. W. (1992) A novel, sensitive, and specific assay for abasic sites, the most commonly produced DNA lesion. Biochemistry, 31, 3703-3708.
32. Atamna, H., Cheung, I. and Ames, B. N. (2000) A method for detecting abasic sites in living cells: age-dependent changes in base excision repair. Proc Natl Acad Sci USA, 97, 686-691.
33. Duthie, S. J. and McMillan, P. (1997) Uracil misincorporation in human DNA detected using single cell gel electrophoresis. Carcinogenesis, 18, 1709-1714.
34. Andersen, S., Ericsson, M., Dai, H. Y., Pena-Diaz, J., Slupphaug, G., Nilsen, H., Aarset, H. and Krokan, H. E. (2005) Monoclonal B-cell hyperplasia and leukocyte imbalance precede development of B-cell malignancies in uracil-DNA glycosylase deficient mice. DNA Repair (Amst), 4, 1432-1441.
35. Laws, G. M., Skopek, T. R., Reddy, M. V., Storer, R. D. and Glaab, W. E. (2001) Detection of DNA adducts using a quantitative long PCR technique and the fluorogenic 5′ nuclease assay (TaqMan). Mutat Res, 484, 3-18.
36. Sikorsky, J. A., Primerano, D. A., Fenger, T. W. and Denvir, J. (2004) Effect of DNA damage on PCR amplification efficiency with the relative threshold cycle method. Biochem Biophys Res Commun, 323, 823-830.
37. Sikorsky, J. A., Primerano, D. A., Fenger, T. W. and Denvir, J. (2007) DNA damage reduces Taq DNA polymerase fidelity and PCR amplification efficiency. Biochem Biophys Res Commun, 355, 431-437.
38. Chen, J., Kadlubar, F. F. and Chen, J. Z. (2007) DNA supercoiling suppresses real-time PCR: a new approach to the quantification of mitochondrial DNA damage and repair. Nucleic Acids Res, 35, 1377-1388.
39. Rothfuss, O., Gasser, T. and Patenge, N. Analysis of differential DNA damage in the mitochondrial genome employing a semi-long run real-time PCR approach. Nucleic Acids Res, 38, e24.
40. Meyer, J. N. QPCR: a tool for analysis of mitochondrial and nuclear DNA damage in ecotoxicology. Ecotoxicology, 19, 804-811.
41. Lee, H. W., Lee, H. J., Hong, C. M., Baker, D. J., Bhatia, R. and O'Connor, T. R. (2007) Monitoring repair of DNA damage in cell lines and human peripheral blood mononuclear cells. Anal Biochem, 365, 246-259.
42. Thomassin, H., Kress, C. and Grange, T. (2004) MethylQuant: a sensitive method for quantifying methylation of specific cytosines within the genome. Nucleic Acids Res, 32, e168.
43. Lasken, R. S., Schuster, D. M. and Rashtchian, A. (1996) Archaebacterial DNA polymerases tightly bind uracil-containing DNA. J Biol Chem, 271, 17692-17696.
44. Rutledge, R. G. and Cote, C. (2003) Mathematics of quantitative kinetic PCR and the application of standard curves. Nucleic Acids Res, 31, e93.
45. Pfaffl, M. W. (2001) A new mathematical model for relative quantification in real-time RT-PCR. Nucleic Acids Res, 29, e45.
46. Wardle, J., Burgers, P. M., Cann, I. K., Darley, K., Heslop, P., Johansson, E., Lin, L. J., McGlynn, P., Sanvoisin, J., Stith, C. M. et al. (2008) Uracil recognition by replicative DNA polymerases is limited to the archaea, not occurring with bacteria and eukarya. Nucleic Acids Res, 36, 705-711.
47. Kidd, E. A., Yu, J., Li, X., Shannon, W. D., Watson, M. A. and McLeod, H. L. (2005) Variance in the expression of 5-Fluorouracil pathway genes in colorectal cancer. Clin Cancer Res, 11, 2612-2619.
48. Kim, N. and Jinks-Robertson, S. (2009) dUTP incorporation into genomic DNA is linked to transcription in yeast. Nature, 459, 1150-1153.

Table 1
TABLE
Uracil content measured from DNA samples synthesized by
Taq polymerase in the presence of different dUTP/dNTP ratio

| | Deoxyuridine measured (1/million dNMP) | | |
|---|---|---|---|
| | Based on ³H- | | |
| | labelled | | |
| dUTP/dNTP | deoxyuridine | Based on real-time PCR reactions | |
| ratio (%) | detection | 544 bp amplicon | 1057 bp amplicon |
| 0 | 0 | 0 | 0 |
| 0.1 | 233 ± 35 | n.d. | 361 ± 2 |
| 0.2 | 488 ± 32 | 146 ± 463 | 933 ± 163 |
| 0.4 | 1125 ± 68 | n.d. | 1465 ± 43 |
| 0.6 | n.d. | 2587 ± 391 | 2070 ± 76 |
| 0.8 | 2205 ± 148 | n.d. | 2570 ± 4 |
| 1 | 2867 ± 693 | 3538 ± 194 | 3806 ± 13 |
| 2 | 6000 ± 563 | 6301 ± 528 | 5166 ± 8 |
| 3 | 8864 ± 426 | 10861 ± 346 | n.d. |
| 4 | 12591 ± 487 | 13107 ± 209 | n.d. |
| 5 | 15966 ± 1065 | 13878 ± 494 | n.d. |

For radioisotopic measurements, ³H labelled dUTP was added to the dNTP pool. First panel: values measured by tritium activity, second panel: values measured by Pfu DNA polymerases amplifying 544 bps in the real-time PCR reaction, third panel: values measured by Pfu DNA polymerases amplifying 1057bps in the real-time PCR reaction. Values represent mean ±standard error of mean, from 3-10 independent measurements, n.d., not determined.

### FIGURE LEGENDS

FIG. 1. Selectivity of Pfu DNA Polymerase for Uracil Bases in DNA

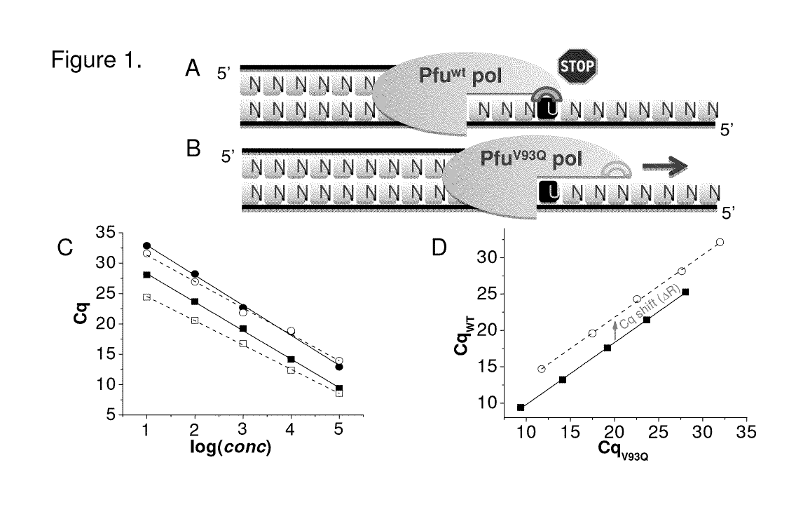

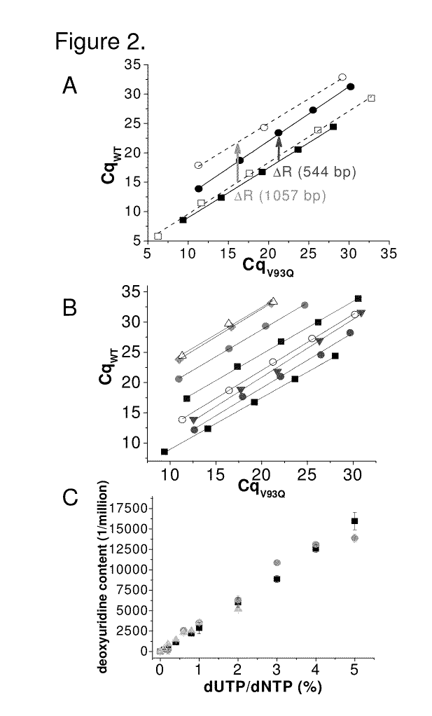

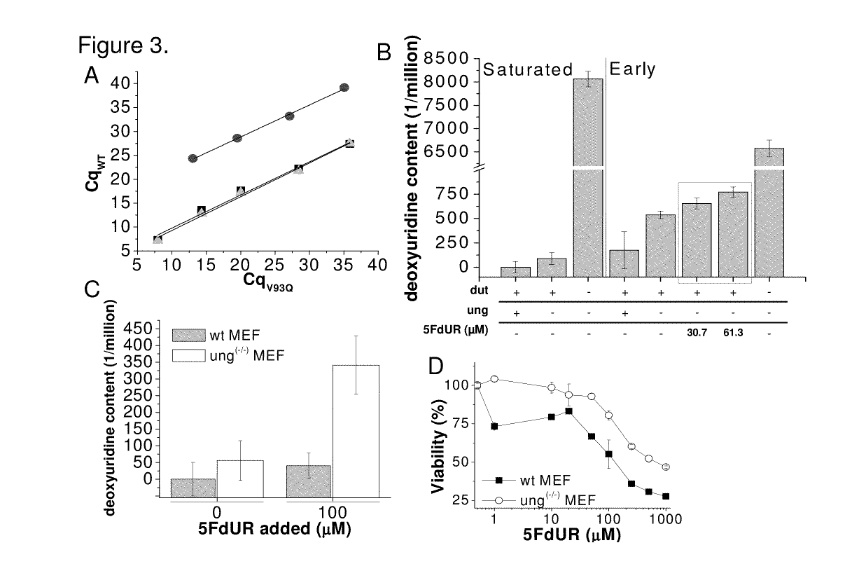

(A) Accommodation of uracil within the specific binding site of Pfu WT-pol stops polymerase action, and the DNA strand cannot be copied further. Only uracil-free templates can be amplified in such a PCR reaction. (B) Lack of uracil recognition within the Pfu V93Q-pol enzyme allows the replication of any DNA templates. (C) Dilution curves of uracil-free (▪ and □) and uracil containing ( and ◯) samples. Logarithm of the relative concentration is showed on the log(conc) axis. Reactions were run using either Pfu WT-pol (empty symbols and dashed curve) or Pfu V93Q-pol (full symbols and solid curve). The Cq shift between the curves corresponds to the difference between the initial template concentration and in case of Pfu WT-pol amplification, the effect of uracil bases in the DNA templates. (D) Plots of the Cq values for Pfu WT-pol against the corresponding Cq values for Pfu V93Q-pol. Plotted values of the uracil-free (▪, solid curve) and the uracil containing (◯, dashed curve) sample dilutions are shown. The curve of uracil containing sample appears at a higher position along the Cq_{WT} axis compared to the curve of the uracil-free sample representing a Cq shift (ΔR).

FIG. 2. Detection of Uracil Bases from Artificial Templates by Pfu DNA Polymerases

(A) Cq_{WT}-C_{V93Q} plots obtained in PCR reactions with Pfu polymerases amplifying 544 (full symbols, solid curves) or 1057 (empty symbols, dashed curves) bp products from DNA sample dilutions. Symbols represents dilution steps of uracil-free reference template (▪ and □) (synthesized by Taq in the presence of 0% dUTP/dNTP ratio) and uracil containing template ( and ◯) (synthesized by Taq in the presence of 0.6% dUTP/dNTP ratio). In case of the 1057 bp amplicon length, a higher ΔR (green arrow) can be observed as compared to that of 544 bp amplicon length (red arrow) allowing more sensitive detection of uracil in the template. (B) Cq_{WT}-Cq_{V93Q} plots obtained in PCR reactions with Pfu polymerases on DNA sample dilutions. DNA samples were synthesized by Taq polymerase in the presence of different concentration ratios of dUTP in the dNTP pool of synthesis mixture: 0% (black squares), 0.2% (red circles), 0.6% (green triangles), 1% (empty circles), 2% (blue squares), 3% (grey circles), 4% (orange diamonds), 5% (empty triangles). Cq shifts (ΔR) of the curves along the Y axis correlates to the higher amount of dUTP added during template synthesis. (C) Comparison of two independent methods to determine uracil content of DNA synthesized by Taq polymerase in the presence of different concentrations of dUTP. Measured deoxyuridine content are shown on the Y axis, dUTP ratios within the dNTP pool in the synthesis mixture are shown on the X axis. Uracil content of DNA was measured through either the radioisotope activity of tritium-labeled deoxyuridine (black squares), or using the Pfu polymerases amplifying 544 bps (orange circles) or 1057 bps (grey triangles) in the real-time PCR reaction.

FIG. 3. Uracil Accumulation Measured in Physiological DNA Samples

(A) Cq_{WT}-Cq_{V93Q} plots obtained in PCR reactions with Pfu WT- and V93Q-pol using dilution steps of plasmid templates. Plasmids were purified from overnight cultures of wild type (black square), ung-151 (grey triangles) and dut-1 ung-1 (red circles) E. coli cells. The curve of the plasmid sample from the dut-1 ung-1 strain is characteristically shifted as compared to the curve of the wt and ung-151 strains. (B) Genomial uracil content measured from E. coli cells in saturated or early exponential (OD=0.5) phase. Early phase ung-151 cells were treated with 30.7 or 61.3 μM 5FdUR. While the genome of the wild type bacteria doesn't show a definite increase in the amount of uracil in early phase as compared to saturated phase, the ung-151 cells accumulate uracil in early phase that can be further increased by treatment with 5FdUR. The strain with dut and ung deficiencies accumulates a large amount of uracil in its genome. (C, D) Wild type and ung^{(-/-)} mouse embryonic fibroblast cell line was treated with 5FdUR. (C) Ung^{(-/-)} MEF cells (white columns) allow a detectable accumulation of uracil in their genome as compared to the wild type MEF cells (grey columns) after 5FdUR treatment. (D) Alamar viability assay of wild type and ung^{(-/-)} MEF cells. Error bars show the standard error of means (SEM) determined from the 3-10 independent measurements.

What is claimed is:

1. A method to quantify uracil content of DNA samples, the method comprising of
(a) selecting a DNA polymerase enzyme that strongly binds to the deaminated base uracil (referred to as enzyme A in this disclosure);
(b) selecting another DNA polymerase enzyme that does not bind strongly to the deaminated base uracil (referred to as enzyme B in this disclosure);
(c) the productivities of enzyme A and enzyme B are compared by measuring the ratio of the products by enzyme A and enzyme B by biochemical methods.

2. As in claim 1, where enzyme A is Pyrococcus furiosus DNA polymerase, while enzyme B is a point mutation of Pyrococcus furiosus DNA polymerase enzyme from valine to glutamine at position 93.

3. As in claim 1, where the productivities of DNA polymerase A and DNA polymerase B are compared by real-time quantitative PCR method.

4. As in claim 2, where the productivities of DNA polymerase A and DNA polymerase B are compared by real-time PCR method.

5. The method of claim 1, applied for quantifying the heterogeneity of uracil distribution within the genome.

6. The method of claim 1, applied for monitoring of the efficacy of anti-cancer chemotherapies in the DNA of cancer cells.

7. The method of claim 1, applied for monitoring the efficacy of cytostatic drugs in oncology.

8. The method of claim 1, applied for monitoring the efficacy of cytostatic drugs in treatment of autoimmune diseases, including, but not limited to rheumatoid arthritis and psoriasis.

9. The method of claim 1, applied for predicting of the efficacy of anti-cancer chemotherapies in the DNA of cancer cells, by applying the chemotherapy drugs in in vitro cell cultures of the patient.

10. The method of claim 1, applied for predicting the efficacy of cytostatic drugs in oncology, by applying the chemotherapy drugs in in vitro cell cultures of the patient.

11. The method of claim 1, applied for predicting the efficacy of cytostatic drugs in treatment of autoimmune diseases, by applying the drugs in in vitro cell cultures of the patient.
